(19) United States
(12) Patent Application Publication
(10) Pub. No.: US 2016/0317360 A1
(43) Pub. Date: Nov. 3, 2016

(54) Foot Wrap

(71) Applicant: Larry Crumbley, Stedman, NC (US)

(72) Inventor: Larry Crumbley, Stedman, NC (US)

(21) Appl. No.: 14/957,132

(22) Filed: Dec. 2, 2015

Related U.S. Application Data
(60) Provisional application No. 62/153,550, filed on Apr. 28, 2015.

(51) Int. Cl. A61F 13/06; A61F 13/00

(52) U.S. Cl. CPC A61F 13/065; A61F 13/00038; A61F 13/00085; A61F 2013/00127; A61F 2013/00272; A61F 2013/0028

(57) ABSTRACT

A foot wrap for relieving foot pain. The foot wrap includes an elongated bandage having a pad thereon. The pad can be placed under the ball of a user's foot and the bandage is then wrapped around the foot in order to secure the pad in the desired position. The ends of the elongated bandage include one or more fasteners thereon, such as hooks, or hook and loop fastening material for securing the bandage in position. Further, an elastic strap is provided so as to be disposed around the user's ankle for further securing the device in position.

### CROSS REFERENCE TO RELATED APPLICATIONS

This application claims the benefit of U.S. Provisional Application No. 62/153,550 filed on Apr. 28, 2015. The above identified patent application is herein incorporated by reference in its entirety to provide continuity of disclosure.

### BACKGROUND OF THE INVENTION

The present invention relates to body wraps. More specifically, the present invention provides a foot wrap with a pad to provide support to the underside of the foot.

Many people suffer from foot pain, specifically on or near the ball of the foot, as the result of nerve damage caused by neuropathy, resulting in impaired sensation and movement. Neuropathy can be caused from systemic diseases, vitamin deficiency, medications, or traumatic injuries, among others. Various remedies exist for relieving pain related to neuropathy, such as oral medications, nerve stimulation therapy, and surgery, among others. However, nerve stimulation therapy and surgery are more expensive and drastic choices when relieving foot and leg pain. Further, continuously taking certain medication has a damaging effect on one's liver.

Alternatively, wearable devices have been disclosed in the prior art that relate to foot wrap devices. These include devices that have been patented and published in patent application publications. These devices generally relate to bandages configured to be wrapped around a user's foot, such as U.S. Patent Application Publication Number 2014/0107554, U.S. Pat. No. 7,856,739, U.S. Patent Application Publication Number 2012/0066815, U.S. Pat. No. 8,240,066, and U.S. Pat. No. 2,572,152.

These prior art devices have several known drawbacks. The devices disclosed in the prior art provide a bandage configured to fit on a user's foot, but fail to provide a pad attached thereto having a shape that corresponds to the ball of the foot.

In light of the devices disclosed in the prior art, it is submitted that the present invention substantially diverges in design elements from the prior art and consequently it is clear that there is a need in the art for an improvement to existing foot wrap devices. In this regard the instant invention substantially fulfills these needs.

### SUMMARY OF THE INVENTION

In view of the foregoing disadvantages inherent in the known types of foot wraps now present in the prior art, the present invention provides a foot wrap wherein the same can be utilized for providing convenience for the user when relieving foot pain.

The foot wrap comprises an elongated bandage having a pad thereon. The pad is configured to be positioned under the ball of a user's foot and the bandage is then wrapped around the foot in order to secure the pad in the desired position. The ends of the elongated bandage include one or more fasteners thereon, such as hooks, or hook and loop fastening material for securing the bandage in position. Further, an elastic strap is provided and is adapted to be disposed around the user's ankle for further securing the device in position.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of an embodiment of the foot wrap.

FIG. 2 shows a perspective view of an embodiment of the foot wrap positioned for attachment onto a user's foot

FIG. 3 shows a perspective view of an embodiment of the foot wrap in use.

### DETAILED DESCRIPTION OF THE INVENTION

Reference is made herein to the attached drawings. Like reference numerals are used throughout the drawings to depict like or similar elements of the foot wrap. The figures are intended for representative purposes only and should not be considered to be limiting in any respect.

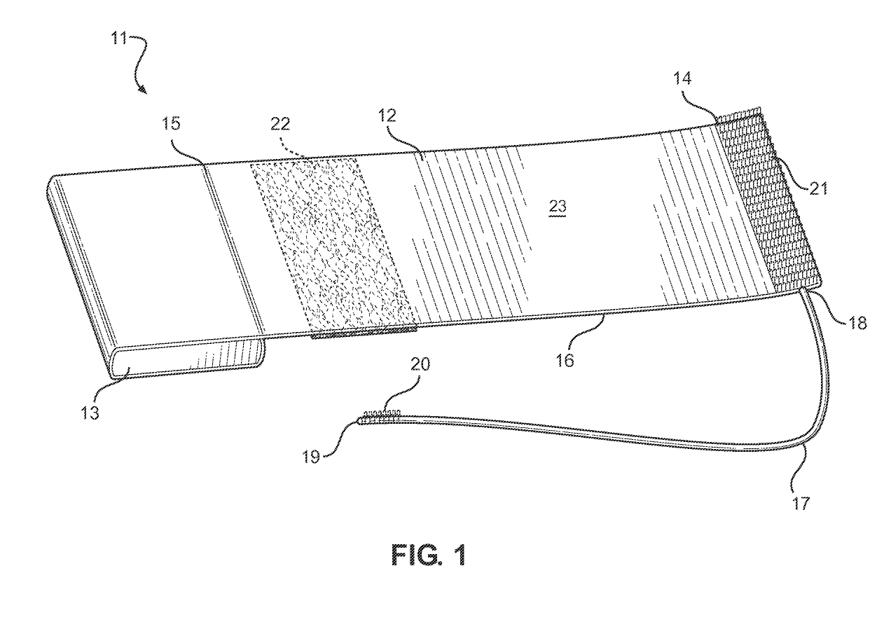

Referring now to FIG. 1, there is shown a perspective view of an embodiment of the foot wrap. The foot wrap 11 comprises an elongated bandage 12 configured to be wrapped around a user's foot. The elongated bandage includes a first end 14 and a second end 15, wherein the second end 15 comprises a pad 13 thereon. In the illustrated embodiment, the elongated bandage 12 is rectangular and composed of a flexible and stretchable material, such as a blend of cotton and polyester, or other suitable materials or blends thereof. The flexible and stretchable material of the elongated bandage 12 allows the user to adjust the length of the bandage 12 in order to fit the size of the user's foot.

The pad 13 is configured to be positioned under the ball of the user's foot. Preferably, the pad 13 is configured to fully cover the ball of a user's foot or correspond to the shape thereof. In the illustrated embodiment, the pad 13 comprises a rectangular shape so as to account for the area of the ball of the foot. In the illustrated embodiment, the pad 13 is composed of foam material, however, in alternate embodiments, the pad 13 is composed of any suitable material configured to rest against the ball of the foot in order to alleviate pressure from nerve pain. Further, the pad 13 is adapted to absorb impact as a user's walks in order to relieve foot pain localized to the ball of the foot.

The elongated bandage 12 allows the pad 13 to be secured in a desired position on the user's foot. Preferably, the width of the pad 13 corresponds to the width of the elongated bandage 12, such that the pad 13 is concealed and there is no excess bandage material protruding from the sides of the foot when the foot wrap 11 is positioned on a user's foot.

Further, the foot wrap 11 comprises one or more fasteners disposed on the elongated bandage 12 and configured to removably secure the bandage in a loop configuration, around the user's foot. In the illustrated embodiment, the fastener is hook material 21 disposed on the front surface 23 of the first end 14 of the elongated bandage 12 and loop material 22 disposed on the rear surface thereof. The area of the loop material 22 is preferably larger in size than that of the area of the hook material 21 in order for the size of the loop to be adjustable based on the size of the user's foot. In this way, the fastener provides another way to allow the foot strap 11 to be adjustable, in addition to the stretchable material of the elongated bandage 12.

In the illustrated embodiment, the foot wrap 11 further comprises a strap 17 having a first end 18 and a second end 19, wherein the strap 17 is configured to be positioned around the ankle of a user for further maintaining the foot wrap 11 in position when worn. The first end 18 of the strap 17 is secured to the elongated bandage 12, preferably on the first end 14 thereof. The second end 19 of the strap 17 is removably securable to the elongated bandage 12 via a fastener 20. In the illustrated embodiment, the second end 19 of the strap 17 comprises loop material thereon such that the second end 19 can be removably secured to the hook material 21 of the elongated bandage 12. However, in other embodiments, the strap 17 is removably securable to the bandage 12 via any suitable fastener, such as a clip or hook.

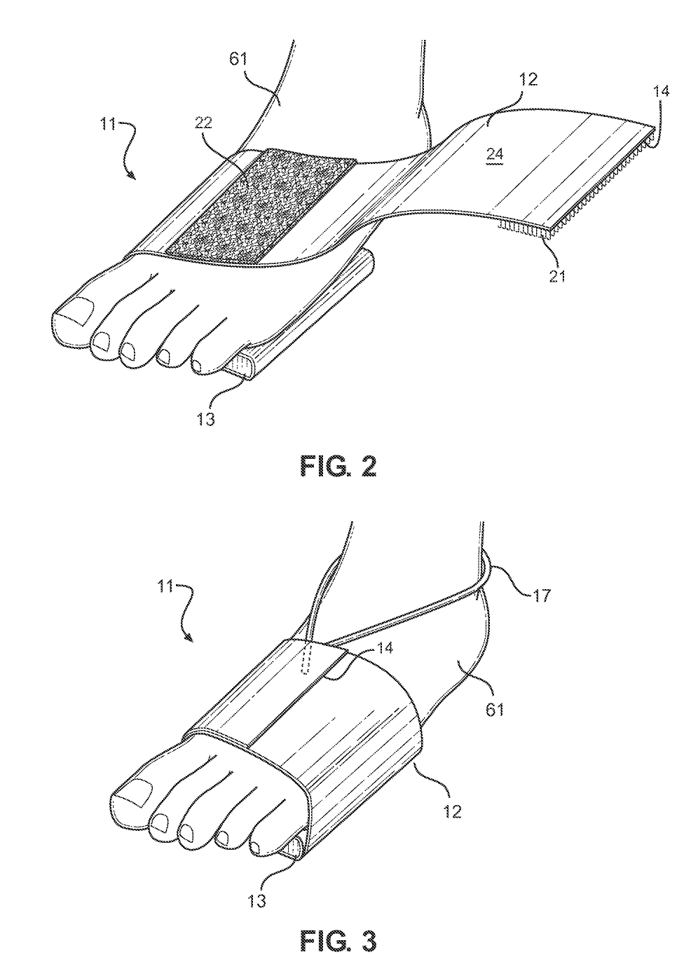

Referring now to FIGS. 2 and 3, there is shown a perspective view of an embodiment of the foot wrap positioned for attachment onto a user's foot and a perspective view of an embodiment of the foot wrap in use, respectively. In operation, the pad 13 is positioned beneath the ball of the foot 61 of the user. The first end 14 of the elongated bandage 12 is wrapped over and around the upper portion of the foot 61. The elongated bandage 12 is configured to be wrapped around the foot one or more times to provide a desired tightness or fit. Once the elongated bandage 12 has formed a loop configuration around the pad 13 and the foot 61, the hook material 21 is removably secured to the loop material 22 disposed on the rear surface 24 of the bandage 12. The second end of the strap 17 is wrapped around the ankle of the user and removably secured to the hook material 21. In this way, the pad 13 is securely placed in the desired position beneath the ball of the user's foot 61, thus, relieving pain as the user sleeps, sits, or walks thereon.

It is therefore submitted that the instant invention has been shown and described in various embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A foot wrap, comprising:
an elongated bandage having a first end and a second end, wherein the elongated bandage is configured to be wrapped around a foot;
a pad disposed on the first end of the elongated bandage and configured to be positioned beneath a ball of the foot;
a fastener disposed on the elongated bandage and configured to removably secure the elongated bandage around the foot;
a strap having a first end and a second end, wherein the first end of the strap is secured to the second end of the elongated bandage and the second end of the strap is removably secured to the second end of the elongated strap, forming a loop configuration.

2) The foot wrap of claim 1, wherein the pad is composed of foam.

3) The foot wrap of claim 1, wherein the pad is rectangular.

4) The foot wrap of claim 1, wherein the fastener comprises hook material disposed on the second end of a front side of the elongated bandage and loop material disposed on a rear side of the elongated bandage.

5) The foot wrap of claim 4, wherein the second end of the strap comprises loop material configured to removably secure to the hook material disposed on the second end of the elongated bandage.

6) The foot wrap of claim 1, wherein the elongated bandage is stretchable.
